(19) United States
(12) Patent Application Publication
(10) Pub. No.: US 2005/0020949 A1
(43) Pub. Date: Jan. 27, 2005

(54) Method of sealing an opening on a waterproof covering for a limb

(76) Inventors: Cheryl Switzer, Wetaskiwin (CA); Terra M. Nielsen, Leduc (CA)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/844,709

(22) Filed: May 13, 2004

(30) Foreign Application Priority Data
Jul. 21, 2003 (CA) 2,435,635

(51) Int. Cl. A61F 5/00

(52) U.S. Cl. 602/3

(57) ABSTRACT

A method of sealing an opening on a waterproof covering for a limb. A first step involves providing a waterproof covering having an opening with a peripheral lip. A second step involves placing a form fitting waterproof elasticised sleeve around a limb above an upper extremity of an affected area to be protected from water on the limb. The elasticised sleeve prevents water from getting to the affected area by passing under the sleeve. A third step involves placing the waterproof covering over the limb with the peripheral lip of the opening encircling the sleeve. A fourth step involves folding an exposed peripheral edge of the sleeve over the peripheral lip. The peripheral lip is sandwiched by the sleeve with the positioning of the sleeve preventing water from entering into the opening.

### FIELD OF THE INVENTION

The present invention relates to a water proof covering which can be used by a person who has an injury while having a shower and, in particular, relates to a method of sealing an opening through which the person extends his or her limb into the waterproof covering.

### BACKGROUND OF THE INVENTION

There are numerous patents relating waterproof coverings used to cover an injury to a limb. The waterproof covering may be placed over a cast, a bandage, stitches, or a recent tattoo. Each patent includes a waterproof covering having an opening into which a person inserts his or her arm or leg. Each patent is distinguishable from the prior art based upon the manner in which the opening is sealed.

U.S. Pat. Des. No. 323,217 (Holden 1992) and U.S. Pat. No. 5,761,746 (Brown 1998) disclose integrally formed elasticised openings. A problem with elasticised openings is that they must be appropriately sized so they are neither so tight as cause discomfort nor so loose as to not make an effective water seal.

U.S. Pat. Nos. 4,254,765 (Brown et al 1981), U.S. Pat. No. 4,911,151 (Rankin et al 1990), U.S. Pat. No. 4,986,265 (Caponi 1991) and U.S. Pat. No. 5,063,919 (Silverberg 1991) disclose separate straps which wrap around the waterproof cover to close the opening. A problem with straps is that care must be taken to avoid folds which may provide channels for water to by pass the sealing area.

U.S. Pat. No. 3,741,203 (Liman 1973), U.S. Pat. No. 4,036,220 (Bellasalma 1977), U.S. Pat. No. 4,363,317 (Broucek 1982), U.S. Pat. No. 4,523,586 (Couri 1985), U.S. Pat. No. 5,342,287 (Jernoiu 1994), U.S. Pat. No. 5,152,282 Elphick et al 1992), U.S. Pat. No. 5,643,183 (Hill 1997) all disclose various configurations of integrally forms straps.

In summary, a problem common to all of these devices is that the elastics or straps used to close the openings must be precisely placed, without folds, and tightly engaged to form an effective water seal. More often than not, all of these conditions are not met and the seal fails.

### SUMMARY OF THE INVENTION

What is required is an alternative method of sealing an opening on a waterproof covering for a limb.

According to the present invention there is provided a method of sealing an opening on a waterproof covering for a limb. A first step involves providing a waterproof covering having an opening with a peripheral lip. A second step involves placing a form fitting waterproof elasticised sleeve around a limb above an upper extremity of an affected area to be protected from water on the limb. The elasticised sleeve prevents water from getting to the affected area by passing under the sleeve. A third step involves placing the waterproof covering over the limb with the peripheral lip of the opening encircling the sleeve. A fourth step involves folding an exposed peripheral edge of the sleeve over the peripheral lip. The peripheral lip is sandwiched by the sleeve with the positioning of the sleeve preventing water from entering into the opening.

The use of a sleeve together with some form of waterproof covering, as described above, provides an effective water seal that will permit an injured person to take a shower without worrying about water infiltrating the waterproof covering and reaching the affected area. Some of the prior art devices were too difficult for an injured person to use without assistance, because the water seal was dependent upon tight and strategically positioned wrapping. In contrast, once the sleeve and waterproof covering are in position, the actual sealing step merely involves folding the sleeve over the peripheral lip of the waterproof covering to seal the opening.

A drawback of some of the prior art devices, is that they were too expensive. In contrast, the sleeve is reusable and can be used with any one of the many types of common household plastic bag. If a specially designed plastic bag were to be used, it is envisaged that there would be advantages if the peripheral lip of the waterproof covering were elasticised. An elasticised peripheral lip would be sure to stay in place while the sleeve was being folded over.

### BRIEF DESCRIPTION OF THE DRAWINGS

These and other features of the invention will become more apparent from the following description in which reference is made to the appended drawings, the drawings are for the purpose of illustration only and are not intended to in any way limit the scope of the invention to the particular embodiment or embodiments shown, wherein:

FIG. 1 is an exploded perspective view of a limb having a cast, along with a water proof covering and a form fitting waterproof elasticised sleeve used to protect the cast from water damage in accordance with the teachings of the present invention.

FIG. 2 is a perspective view of the form fitting waterproof elasticised sleeve illustrated in FIG. 1, positioned on the limb.

FIG. 3 is a perspective view of the water proof covering illustrated in FIG. 1, covering the limb and positioned around the form fitting waterproof elasticised sleeve illustrated in FIG. 2.

FIG. 4 is a side elevation view, in section, of the form fitting waterproof elasticised sleeve folded over the peripheral lip of the waterproof covering to seal the opening in accordance with the teachings of the present invention.

FIG. 5 is a side elevation view, in section, of a variation on the preferred method with the form fitting waterproof elasticised sleeve overlying the peripheral lip of the waterproof covering to seal the opening.

### DETAILED DESCRIPTION OF THE PREFERRED EMBODIMENT

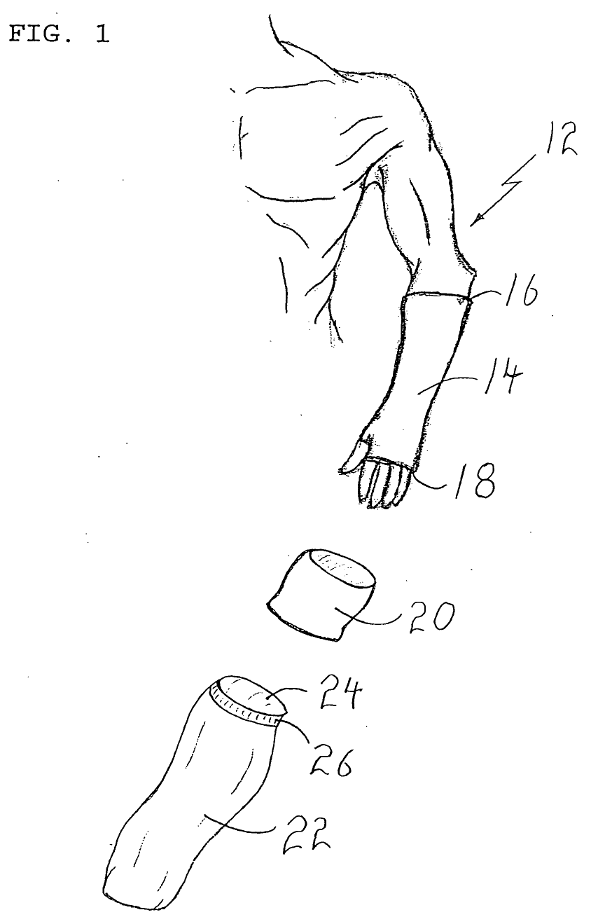

The preferred method of sealing an opening on a waterproof covering for a limb will now be described with reference to FIGS. 1 through 5.

Referring to FIG. 1, there is illustrated a limb 12 having a cast 14. Cast 14 has been used as a concrete example of a typical affected area that one might wish to shield from water on a limb. It will be appreciated that there are other types of wounds, such as tensor bandages, stitches, or tattoos. It will also be appreciated that there may be reasons to keep an affected area dry on a limb, that have nothing to do with to wounds or injuries of any kind. The affected area marked by cast 14 has an upper extremity 16 and a lower extremity 18. There is also illustrated a form fitting waterproof elasticised sleeve 20 and a waterproof covering 22. There will now be described how the cast is protected from water using form fitting waterproof elasticised sleeve 20 and waterproof covering 22 in accordance with the teachings of the present invention.

Referring to FIG. 1, in accordance with the teachings of the present invention, a first step involves providing a waterproof covering 22 having an opening 24 with a peripheral lip 26. In the illustrated embodiment, waterproof covering 22 is a plastic bag and peripheral lip 26 is elasticised.

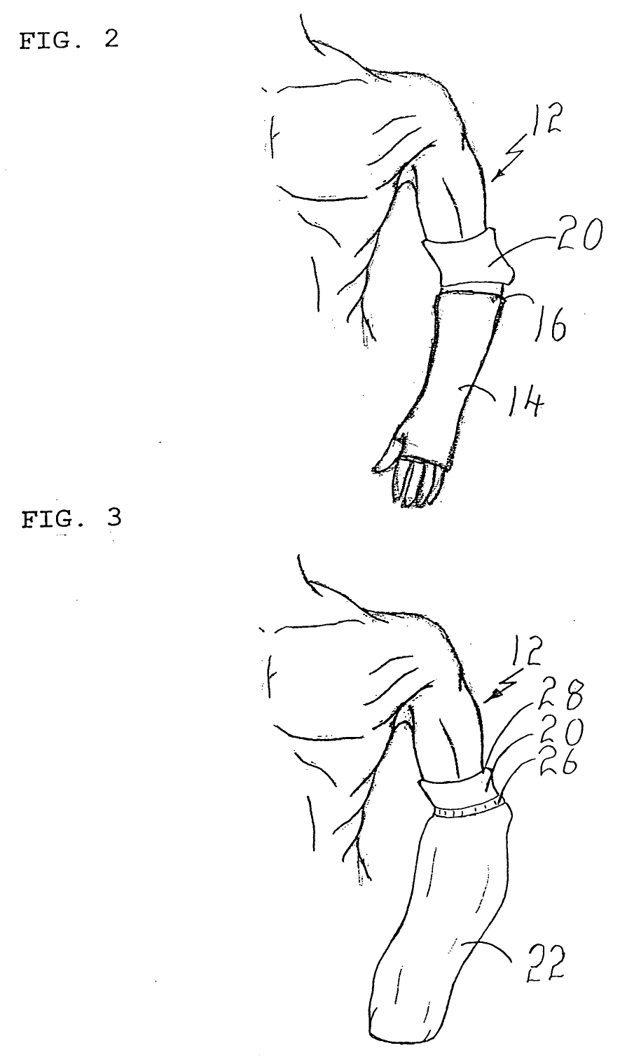

Referring to FIG. 2, a second step involves providing a form fitting waterproof elasticised sleeve 20 and placing it around limb 12 above upper extremity 16 of cast 14 such that water is prevented from passing under form fitting waterproof elasticised sleeve 20.

Referring to FIG. 3, a third step involves placing waterproof covering 22 over limb 12 with peripheral lip 26 encircling a portion of form fitting waterproof elasticised sleeve 20, leaving an exposed peripheral edge 28 of form fitting waterproof elasticised sleeve 20.

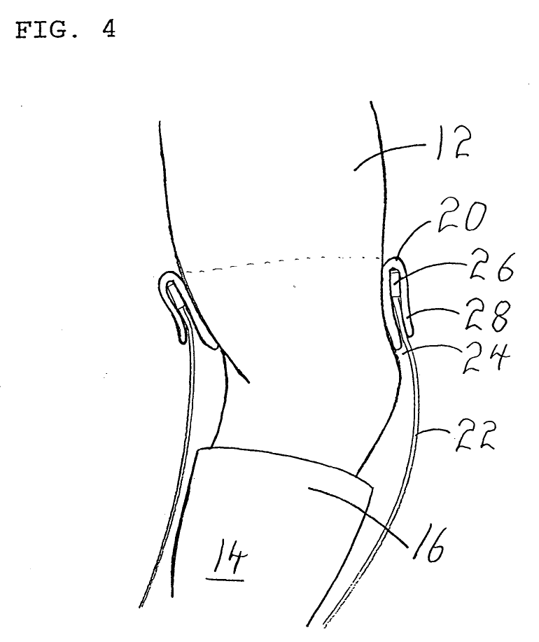

Referring to FIG. 4, a fourth step involves folding exposed peripheral edge 28 of form fitting waterproof elasticised sleeve 20 over peripheral lip 26 such that peripheral lip 26 is sandwiched by form fitting waterproof elasticised sleeve 20 and, in turn, preventing water from entering into opening 24.

Advantages:

The method described above is considered to provide the following advantages:
the use of a form fitting waterproof elasticised sleeve (hereinafter referred to as “the sleeve”) is easy to apply and remove by an injured person;
the sleeve can be readjusted as required during use;
the sleeve provides an effective water seal;
the sleeve allows the user increased freedom of movement;
the sleeve does not require an exact or an uncomfortably tight engagement to make an effective seal;
the sleeve and its associate waterproof covering can be made from inexpensive materials;
the sleeve is reusable.

Variations:

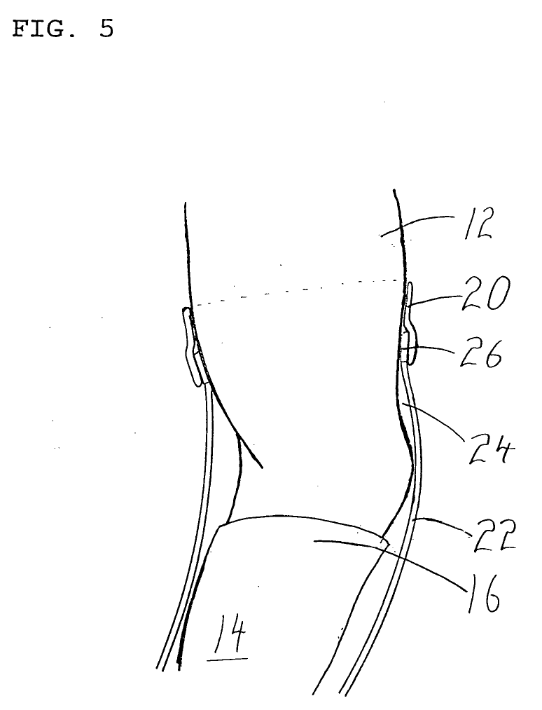

Referring to FIG. 5, limited success has been obtained through a variation of the above described method using form fitting waterproof elasticised sleeve 20 and a waterproof covering 22. In accordance with this variation on the method waterproof covering 22 is placed over an affected area 14 to be protected from water on a limb 12 first; with the peripheral lip 26 of the opening 24 encircling the limb above an upper extremity 16 of affected area 14. Form fitting waterproof elasticised sleeve 20 is then placed around limb 12 overlying peripheral lip 26 of opening 24. This method works as sleeve 20 prevents water from getting to affected area 14 by either passing under sleeve 20 or by entering into opening 24 of waterproof covering 22. In working with this method, it was found that it was very difficult for an injured person to put sleeve 20 into the correct position without assistance. For this reason, placing sleeve 20 on first, placing waterproof covering 22 on second and folding sleeve 20 back over opening 24 on waterproof covering 22 is preferred.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from the spirit and scope of the invention as hereinafter defined in the claims.

1. A method of sealing an opening on a waterproof covering for a limb, comprising the following steps:
providing a waterproof covering having an opening with a peripheral lip;
placing a form fitting waterproof elasticised sleeve around a limb above an upper extremity of an affected area to be protected from water on the limb, the elasticised sleeve preventing water from getting to the affected area by passing under the sleeve;
placing the waterproof covering over the limb with the peripheral lip of the opening encircling the sleeve; and
folding an exposed peripheral edge of the sleeve over the peripheral lip, such that the peripheral lip is sandwiched by the sleeve with the positioning of the sleeve preventing water from entering into the opening.

2. The method as defined in claim 1, wherein the waterproof covering is a plastic bag.

3. The method as defined in claim 1, wherein the peripheral lip of the waterproof covering is elasticised.

4. In combination:
a limb having an affected area to be protected from water, the affected area having an upper extremity and a lower extremity;
a form fitting waterproof elasticised sleeve positioned around the limb above the upper extremity of the affected area, the elasticised sleeve preventing water from getting to the affected area by passing under the sleeve;
a waterproof covering having an opening with a peripheral lip, the waterproof covering being positioned over the limb with the peripheral lip of the opening encircling the sleeve; and
an exposed peripheral edge of the sleeve being folded over the peripheral lip, such that the peripheral lip is sandwiched by the sleeve with the positioning of the sleeve preventing water from entering into the opening.

5. The combination as defined in claim 4, wherein the waterproof covering is a plastic bag.

6. The combination as defined in claim 4, wherein the peripheral lip of the waterproof covering is elasticised.

7. A method of sealing an opening on a waterproof covering for a limb, comprising the following steps:
providing a waterproof covering having an opening with a peripheral lip;
placing the waterproof covering over an affected area to be protected from water on a limb with the peripheral lip of the opening encircling the limb above an upper extremity of the affected area; and
placing a form fitting waterproof elasticised sleeve around the limb overlying the peripheral lip of the opening, the elasticised sleeve preventing water from getting to the affected area by either passing under the sleeve or by entering into the opening of the waterproof covering.
